(19) United States
(12) Patent Application Publication
(10) Pub. No.: US 2024/0164792 A1
(43) Pub. Date: May 23, 2024

(54) RESECTION TEMPLATE FOR THE RESECTION OF A BONE

(71) Applicant: Karl Leibinger Medizintechnik GmbH & Co. KG, Mühlheim / Donau (DE)

(72) Inventors: Ralf Leibinger, Mühlheim (DE); Artur Kühn, Radolfzell (DE); Frank Reinauer, Emmingen-Liptingen (DE)

(21) Appl. No.: 18/511,545

(22) Filed: Nov. 16, 2023

(30) Foreign Application Priority Data
Nov. 18, 2022 (DE) 10 2022 212 308.5

(51) Int. Cl. A61B 17/15

(52) U.S. Cl. CPC A61B 17/15

(57) ABSTRACT

The invention provides a resection template for the resection of a bone. The resection template is equipped with a plate-shaped base part (1) having a front side (VS) and a rear side (RS), wherein the rear side (RS) has a bearing face (10) for bearing on the bone, wherein the front side (VS) has a fastening flange (2) which extends away from the front side (VS) and is arranged next to an end edge (K) of the base part (1) or a slot of the base part (1), a cutting guide attachment (20) which can be fitted onto the fastening flange (2) from a side (S2) of the fastening flange (2) opposite to the front side (VS), wherein the cutting guide attachment (20) has a saw slot (201) or a saw abutment for guiding a sawing tool, which slot or abutment is arranged along the end edge (K) of the base part (1) or along the slot of the base part (1) when the cutting guide attachment (20) is fitted on.

The present invention relates to a resection template for the resection of a bone.

Although it may be used for any bones, the present invention and its underlying problem will be explained with the aid of bones in the craniomaxillofacial region and bone extremities.

Resection templates may generally be used in all surgical osteotomies, but are particularly often used in the reconstruction of the lower jaw with an autologous bone transplant (fibula or iliac crest) or in the resection of meningiomas.

Despite the advances in microvascular reconstruction techniques, it is still a demanding task to return a bone, for example the lower jawbone, into its original anatomical shape and to restore functional and aesthetic aspects. The use of a fibula from the lower leg of the patient has become established as a proven method for restoration. Other donor regions may however also be mentioned in the context of a transplant, for example taking bone segments from the iliac crest or scapula.

Product improvements in recent years have primarily been aimed at computer-assisted approaches in combination with patient-specifically manufactured resection templates and implants, with the aid of which the preoperative planning can be transferred to real-time surgery.

Maximally precise osteotomy/resection on the transplant/resectate to be used is crucial for correct implementation of this planning during the operation. This may only be achieved with defined cut guiding and exact cutting faces on the cutting guide.

The previous methods have very largely been based on the principle of a metal inlay, which is fitted into a plastic border. This, however, results in a correspondingly largely dimensioned module which requires a very large wound opening and therefore represents considerable stress for the patient, since the insertion of the inlay takes place laterally.

EP 3 451 944 A1 discloses a fibula bone material removal and transfer template comprising a central part which has a central body, at the ends of which there is respectively a bone separating tool guide section, at least one of the bone separating tool guide sections being mounted so that it can be moved away from and towards the central part.

DE 10 2016 108 433 A1 discloses a lower jaw resection template comprising a central component which is adapted for application on a segment, for instance a symphysis segment, of a jawbone, for instance a lower jaw or an upper jaw, there being at least two separating tool guide sections on the central component, there being a positioning aid between the two or more separating tool guide sections in order to achieve spatially correct alignment of the lower jaw resection template with the jawbone.

US 2021/0137537 A1 discloses a resection template comprising a cutting guide, which defines a plurality of cutting slots and comprises at least one removable insert, which is positioned between the plurality of cutting slots, the at least one removable insert defining an opening and being designed to receive a surgical fastening device; and a template guide which defines a feature, the at least one removable insert being configured so that it is fitted at least partially into the template guide by using the feature.

Further resection templates are known from WO 2017/162444 A1 and EP 3 470 002 A1.

The object of the present invention is to provide an improved resection template for the resection of a bone, which is better accessible intraoperatively and has a smaller space requirement.

The object is achieved by a resection template for the resection of a bone having the features of claim 1, that is to say by a resection template for the resection of a bone, comprising a plate-shaped base part having a front side and a rear side, wherein the rear side has a bearing face for bearing on the bone, wherein the front side has a fastening flange which extends away from the front side and is arranged next to an end edge of the base part or a slot of the base part; a cutting guide attachment which can be fitted onto the fastening flange from a side of the fastening flange opposite to the front side; wherein the cutting guide attachment has a saw slot or a saw abutment for guiding a sawing tool, which slot or abutment is arranged along the end edge of the base part or along the slot of the base part when the cutting guide attachment is fitted on.

According to the concept on which the invention is based, the reception is produced no longer as a border or frame on a supporting body, but by a preferably slender fastening flange starting from the upper side, for example a plate flange or spiked flange, which allows the entire design in a much smaller overall size. It is therefore possible to operate with a significantly smaller wound opening, which is less traumatic for the patient.

The invention is based on a base body consisting of a material which is compatible with the body, for example a plastic, and a cutting guide attachment with defined positioning and cut guiding, consisting of a strong material, for example a metal, an open or closed variant being possible for the cutting guide attachment. In contrast to previously known systems, the invention is not designed as an inserted part but is configured as a cutting guide attachment for a fastening flange.

The base body is placed onto the bone to be cut and is fastened, for example screwed, thereon. The cutting guide attachment is then fitted by the surgeon onto the fastening flange intended therefor. The handling is performed very simply with the thumb and index finger. A preferably encoded form fit allows only one positioning, which is predetermined by the planning. The more strongly the cutting guide attachment is pressed onto the base part, the more self-retention this combination experiences. This may be achieved by an optional chamfer on the fastening flange, which is clamped in the parallel-running guide of the cutting guide attachment. Furthermore, the fastening displaced for the osteotomy generates a moment which tilts the guide and secures it against loss—which occurs due to the saw and vibrations.

The nature of the design offers a significant reduction of the overall size, which represents an advantage for the user in comparison with the previously known methods, but also (above all) for the patient. The simple design allows easy handling.

Preferred developments of the subjects according to the invention may be found in the dependent claims and the description, in particular with reference to the drawings.

According to one preferred embodiment, the fastening flange is formed as a plate flange. Such a configuration is stable and requires little space.

According to another preferred embodiment, the cutting guide attachment is formed as a ring profile, which has a rectilinear region in which the saw slot is arranged or on which the saw abutment is arranged, wherein there is a first end collar region at the first end of the rectilinear region and a second end collar region at the second end of the rectilinear region, wherein the first and second end collar regions together with the rectilinear region form a reception region for the plate flange, and wherein the first and second end collar regions are formed in such a way that they enclose the plate flange on its opposite edge regions.

According to another preferred embodiment, there is an open region of the ring profile between the first and second end collar regions.

According to another preferred embodiment, the plate flange has a first abutment ridge and a second abutment ridge, which are arranged in such a way that they form a respective lateral abutment for the first and second end collar regions. This allows encoding to prevent fitting on incorrectly.

According to another preferred embodiment, the plate flange has an abutment block, which is arranged in such a way that it forms a respective lateral abutment for the first and second end collar regions. This likewise allows encoding to prevent fitting on incorrectly.

According to another preferred embodiment, the plate flange has on its opposite edges a respective first and second broadening region, which widen in the direction from the side of the plate flange opposite to the front side in the direction of the front side, so that an increasing clamp effect can be exerted on the ring profile when fitting on. This ensures stable holding of the ring profile.

According to another preferred embodiment, the plate flange has on its opposite edges a respective first and second end abutment region, which limit the fitting depth of the ring profile onto the plate flange.

According to another preferred embodiment, the first and second end collar regions have a respective gripping rib region. This allows secure handleability.

According to another preferred embodiment, the plate flange has a first slot and a second slot starting from the side of the plate flange opposite to the front side, which end in a respectively associated first broadening region and second broadening region, and wherein the first and second end collar regions have a first and second locking bolt, which can be introduced through the associated slot into the first and second broadening regions in order to lock the ring profile.

According to another preferred embodiment, the plate flange has a first slot and a second slot starting from the side of the plate flange opposite to the front side, which end in a respectively associated first broadening region and second broadening region, and wherein the rectilinear region has a first and second locking bolt, which can be introduced through the associated slot into the first and second broadening regions in order to lock the ring profile.

According to another preferred embodiment, the fastening flange is formed as a spiked flange having one or more spikes.

According to another preferred embodiment, the cutting guide attachment is formed as a ring profile which has one or more spike reception regions, according to the number of spikes of the spiked flange.

According to another preferred embodiment, the plate flange has a groove on the side of the plate flange opposite to the front side, wherein the cutting guide attachment has a connecting region, which can be inserted into the groove, starting from the rectilinear region, and wherein a grip region is provided on the region of the connecting region opposite to the rectilinear region. The two grips may furthermore be arranged somewhat obliquely in order to achieve a clamping effect/fixing. Owing to the flexibility of the plastic, the cutting guide attachment may then be removed by pressing the grips together.

According to another preferred embodiment, the plate-shaped base part has a multiplicity of through-holes, which are used to receive fastening means, in particular screws or nails, for fastening on the bone.

According to another preferred embodiment, the plate-shaped base part with the fastening flange is formed from a plastic material and the cutting guide attachment is formed from a metallic material.

According to another preferred embodiment, the cutting guide attachment is formed in such a way that the upper side and the lower side of the cutting guide attachment are arranged plane-parallel, so that the cutting depth of a saw tool fitted on is constant over the entire lateral extent of the cutting guide attachment.

According to another preferred embodiment, the upper side of the cutting guide attachment has a curved contour, the effect of which is that the cutting depth of a saw tool fitted on varies over the lateral extent of the cutting guide attachment.

According to another preferred embodiment, the curved contour of the upper side of the cutting guide attachment is curved in such a way that it replicates or at least locally approximates a contour of the underlying bone to be resected. In this way, particular tissue structures which surround the bone to be resected may be protected by the curvature, since they cannot be reached by the saw tool.

The configurations and developments above may, insofar as is feasible, be combined with one another in any desired way. Further possible configurations, developments and implementations of the invention also include not explicitly mentioned combinations of features of the invention which are described above or below in relation to the exemplary embodiments.

The invention will be explained in more detail below with the aid of exemplary embodiments in the figures of the drawing. Sometimes in a schematic representation:

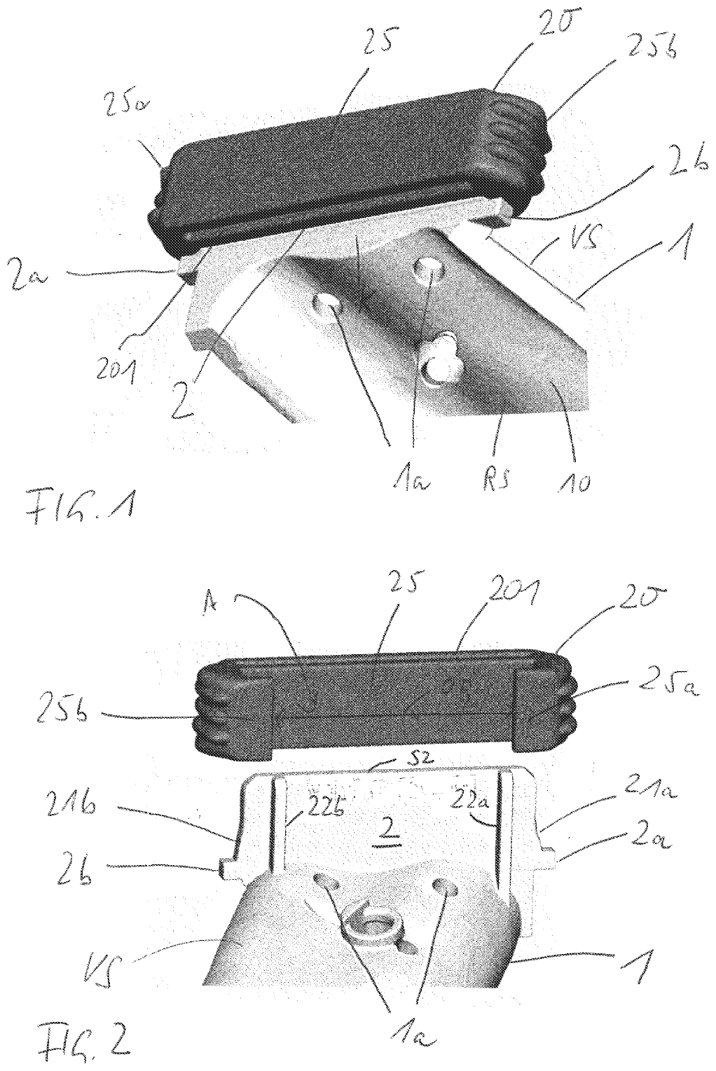

FIG. 1 shows a partial perspective representation of a resection template according to a first embodiment of the present invention;

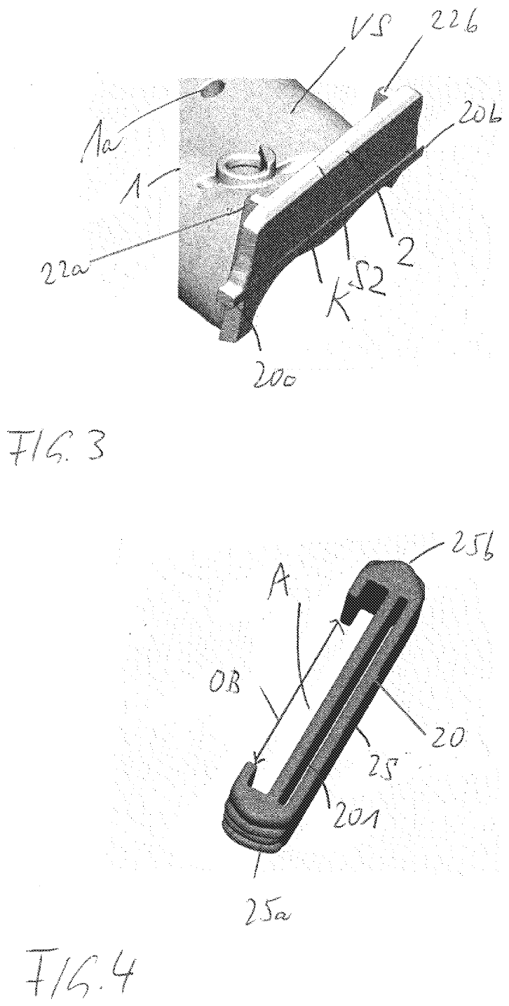

FIG. 2 shows a partial perspective exploded view of the resection template according to the first embodiment of the present invention;

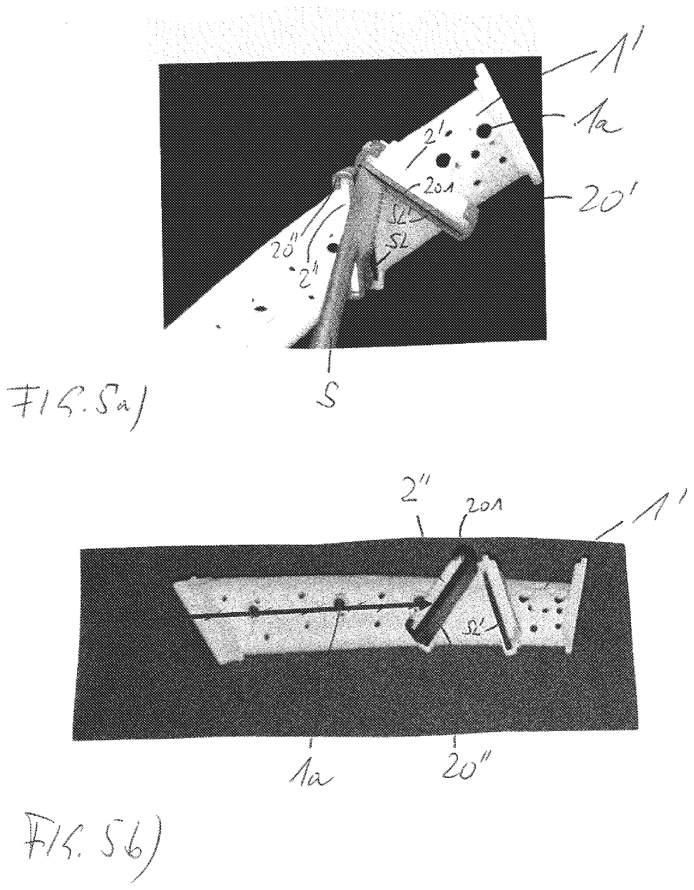

FIG. 3 shows a partial perspective representation of the base part of the resection template according to the first embodiment of the present invention;

FIG. 4 shows a perspective representation of the cutting guide attachment of the resection template according to the first embodiment of the present invention;

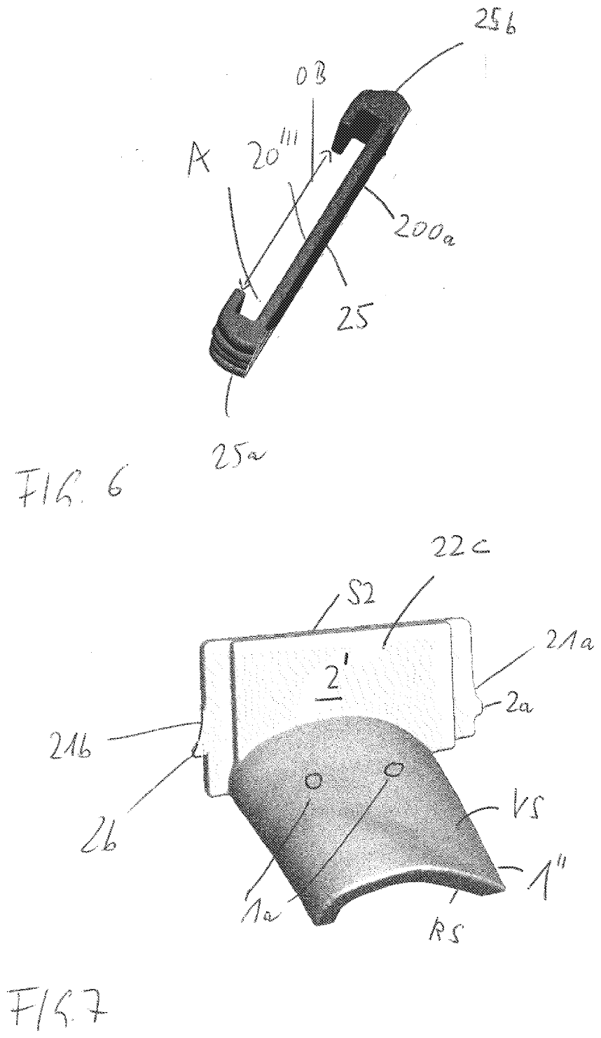

FIG. 5a), b) show perspective representations of a resection template according to a second embodiment of the present invention, specifically with FIG. 5a) showing a partial perspective representation with a saw tool fitted and FIG. 5b) showing a perspective representation with only a cutting guide attachment fitted on;

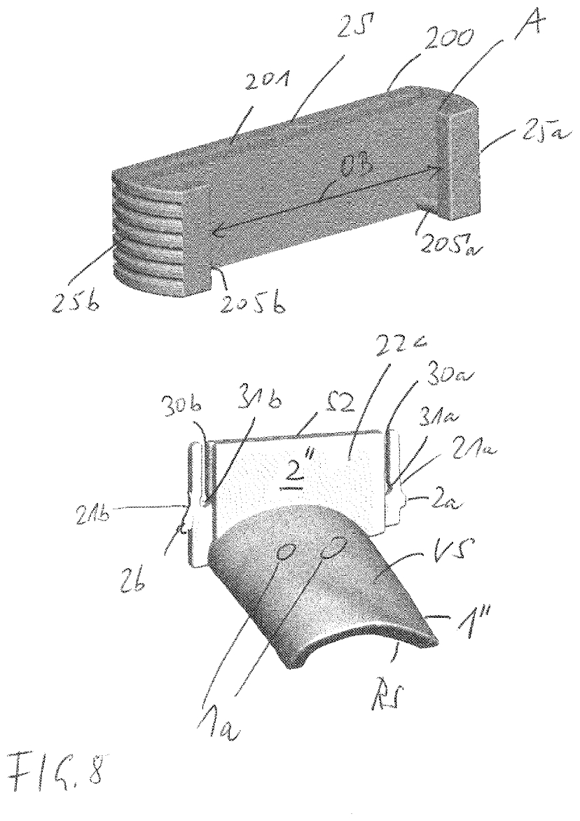

FIG. 6 shows a perspective representation of a cutting guide attachment of a resection template according to a third embodiment of the present invention;

FIG. 7 shows a perspective representation of a base part of a resection template according to a fourth embodiment of the present invention;

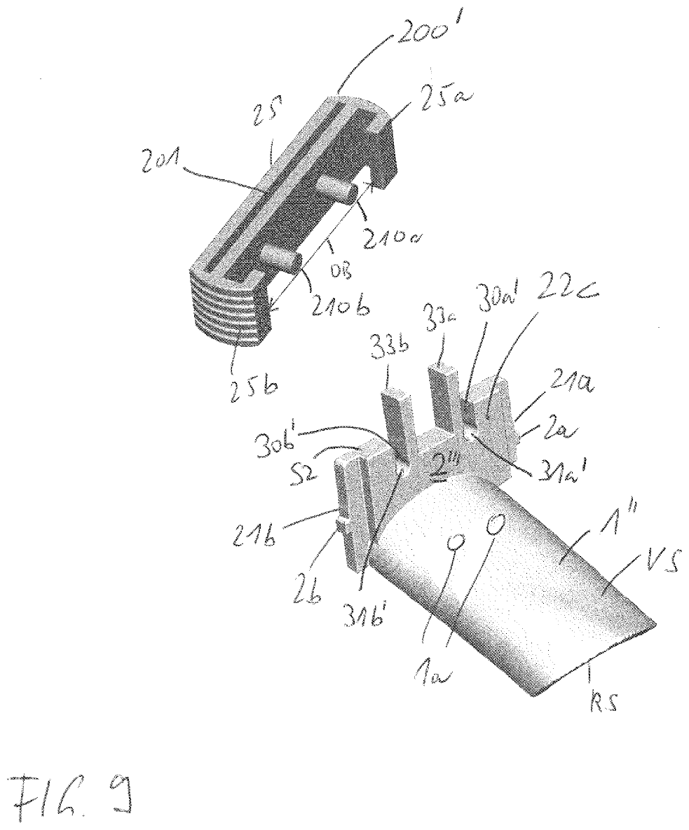

FIG. 8 shows a partial perspective exploded view of a resection template according to a fifth embodiment of the present invention;

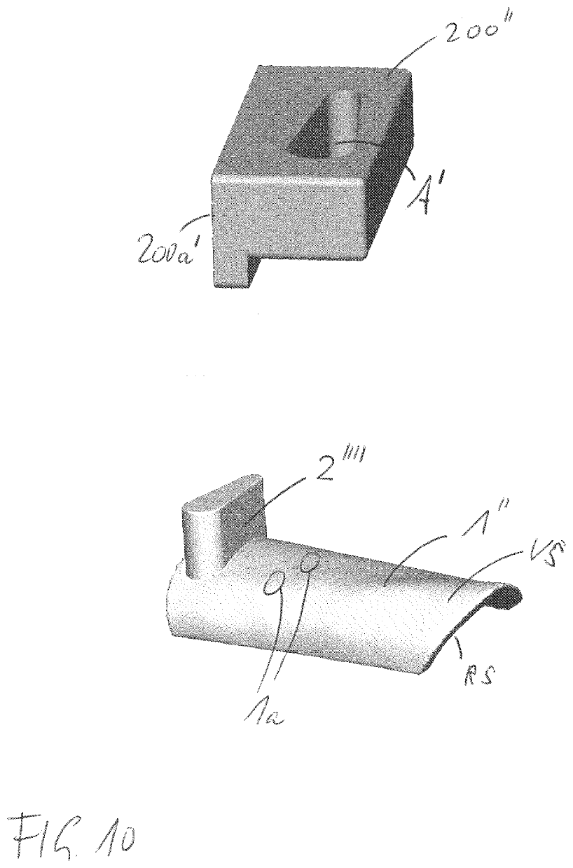

FIG. 9 shows a partial perspective exploded view of a resection template according to a sixth embodiment of the present invention;

FIG. 10 shows a partial perspective exploded view of a resection template according to a seventh embodiment of the present invention;

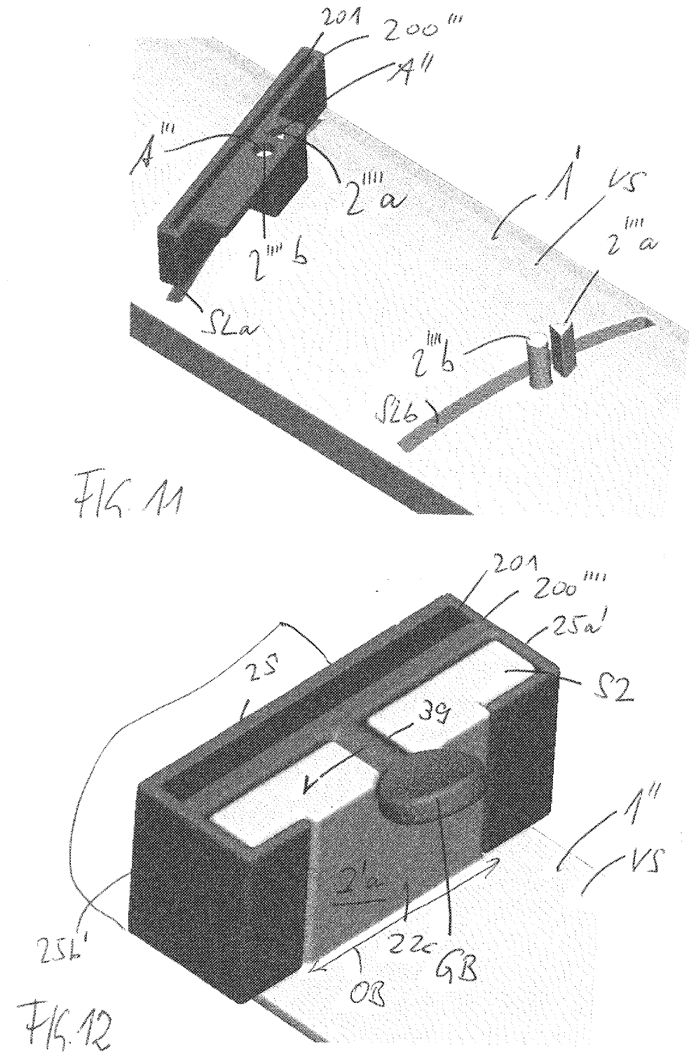

FIG. 11 shows a partial perspective representation of a resection template according to an eighth embodiment of the present invention;

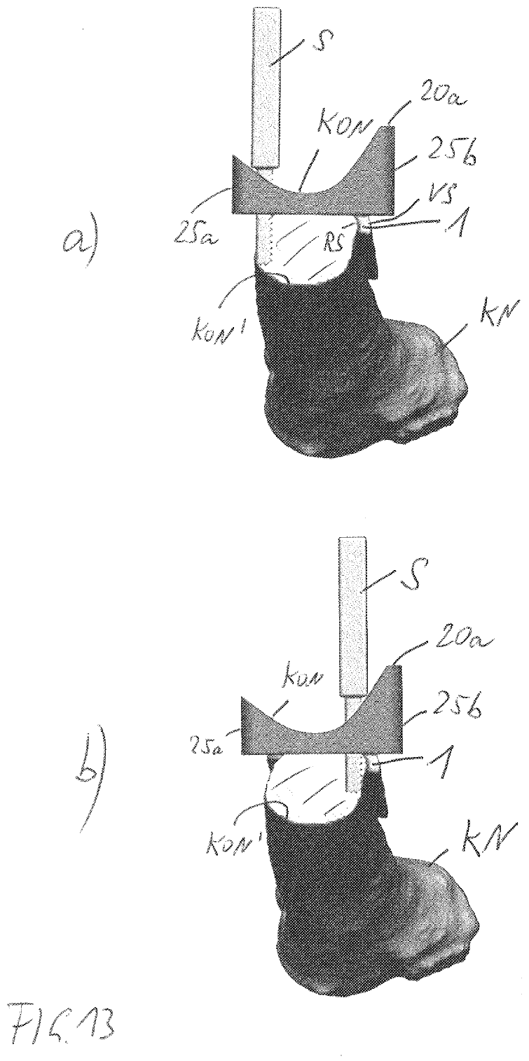

FIG. 12 shows a partial perspective representation of a resection template according to a ninth embodiment of the present invention; and

FIG. 13a), b) show perspective representations of a resection template according to a tenth embodiment of the present invention, specifically with FIG. 13a) showing a partial perspective representation with a fitted saw tool in a first position of the saw tool and FIG. 13b) showing a partial perspective representation with a fitted saw tool in a second position of the saw tool.

In all the figures, elements and devices which are the same or functionally equivalent—unless otherwise indicated—have been provided with the same reference signs.

FIG. 1 shows a perspective representation of a resection template according to a first embodiment of the present invention.

The resection template for the resection of a bone according to the first embodiment has a plate-shaped base part 1 with a front side VS and a rear side RS. It is made from a plastic material.

A multiplicity of through-holes 1a which are used to receive fastening means, in particular screws or nails, for fastening on the bone, are provided in the base part 1.

The rear side RS has a bearing face 10 for bearing on the bone, the contour of which is matched to the bone to be processed.

The front side VS has a fastening flange 2 which extends away from the front side VS and is formed integrally with the base part, and which in this embodiment is arranged next to an end edge K of the base part 1.

A cutting guide attachment 20 formed from a metallic material is fitted in a clamping fashion onto the fastening flange 2 from a side S2 of the fastening flange 2 opposite to the front side VS (cf. FIG. 2).

The cutting guide attachment 20 has a saw slot 201, which is arranged along the end edge K of the base part 1, for guiding a sawing tool.

FIG. 2 shows a partial perspective exploded view of the resection template according to the first embodiment of the present invention, FIG. 3 shows a partial perspective representation of the base part of the resection template according to the first embodiment of the present invention, and FIG. 4 shows a perspective representation of the cutting guide attachment of the resection template according to the first embodiment of the present invention.

Still with reference to FIGS. 1 to 4, the fastening flange 2 is formed as a relatively narrow plate flange.

The cutting guide attachment 20 is formed as an open ring profile, which has a rectilinear region 25 in which the saw slot 201 is arranged, there being a first end collar region 25a at the first end of the rectilinear region 25 and a second end collar region 25b at the second end of the rectilinear region 25, the first and second end collar regions 25a, 25b together with the rectilinear region 25 forming a reception region A for the plate flange. For this purpose, the first and second end collar regions 25a, 25b enclose the plate flange on its opposite edge regions. Between the first and second end collar regions 25a, 25b, there is an open region OB of the ring profile. The first and second end collar regions 25a, 25b have a respective gripping rib region, which allows simple insertion by the surgeon using the thumb and index finger.

The plate flange furthermore has a first abutment ridge 22a and a second abutment ridge 22b, which are arranged in such a way that they form a respective lateral abutment for the first and second end collar regions 25a, 25b. The first and second abutment ridges 22a, 22b therefore form encoding which prevents the ring profile from being fitted on incorrectly.

The plate flange has on its opposite edges a respective first and second broadening region 21a, 21b, which widen in the direction from the side S2 of the plate flange opposite to the front side VS in the direction of the front side VS, so that an increasing clamp effect can be exerted on the ring profile when fitting on.

On the opposite edges of the plate flange, a respective lower first and second end abutment region 2a, 2b are provided, which limit the fitting depth of the ring profile onto the plate flange.

FIG. 5a), b) show perspective representations of a resection template according to a second embodiment of the present invention, specifically with FIG. 5a) showing a partial perspective representation with a saw tool fitted and FIG. 5b) showing a perspective representation with only a cutting guide attachment fitted on.

The second embodiment differs from the first embodiment in respect of the formation of the base part 1′. The base part 1′ has a first and second fastening flange 2′, 2″, which are arranged along a respective first and second slot SL, SL′ of the base part 1′. Correspondingly provided are a first and second cutting guide attachment 20′, 20″, which are fitted onto the first and second fastening flanges 2′, 2″ in such a way that their respective saw slot 201 is arranged along the first and second slot SL, SL′ of the base part 1′.

FIG. 5a) additionally represents a saw tool S fitted in the saw slot 201 of the first cutting guide attachment 20′.

In other regards, the second embodiment is formed in entirely the same way as the first embodiment.

FIG. 6 shows a perspective representation of a cutting guide attachment of a resection template according to a third embodiment of the present invention.

The representation of FIG. 6 is similar to the representation of FIG. 4, and differs from FIG. 4 in respect of the configuration of the cutting guide attachment.

In particular, instead of the saw slot 201, the cutting guide attachment 20″ according to FIG. 6 has a saw abutment 200a for guiding a sawing tool, which is arranged along the end edge K of the base part 1 or along the respective slot SL, SL′ of the base part 1′ when the cutting guide attachment 20″ is fitted on.

In other regards, the third embodiment is formed in entirely the same way as the first embodiment.

FIG. 7 shows a perspective representation of a base part of a resection template according to a fourth embodiment of the present invention.

The representation of FIG. 7 is similar to the representation of FIG. 2 or FIG. 3, and differs from FIG. 2 or FIG. 3 in respect of the configuration of the fastening flange 2′.

In particular, the fastening flange 2′ has a continuous abutment block 22c, which is arranged in such a way that it forms a respective lateral abutment for the first and second end collar regions 25a, 25b.

In other regards, the fourth embodiment is formed in entirely the same way as the first embodiment.

FIG. 8 shows a partial perspective exploded view of a resection template according to a fifth embodiment of the present invention.

The representation of FIG. 8 is similar to the representation of FIG. 2, and differs from FIG. 2 in respect of the configuration of the cutting guide attachment 200 and of the fastening flange 2″ as well as the contour of the base part 1″.

The fastening flange 2″ has a first slot 30a and a second slot 30b starting from the side S2 of the fastening flange 2″ opposite to the front side VS, which end in a respectively associated first broadening region 31a and second broadening region 31b.

The first and second end collar regions 25a, 25b of the cutting guide attachment 200 have a first and second locking bolt 205a, 205b, which can be introduced through the associated slot 30a, 30b into the first and second broadening regions 31a, 31b in order to lock the cutting guide attachment 200.

In other regards, the fifth embodiment is formed in entirely the same way as the first embodiment.

FIG. 9 shows a partial perspective exploded view of a resection template according to a sixth embodiment of the present invention.

The representation of FIG. 9 is similar to the representation of FIG. 2, and differs from FIG. 2 in respect of the configuration of the cutting guide attachment 200′ and of the fastening flange 2′″ as well as the contour of the base part 1″.

The fastening flange 2′″ has a first slot 30a′ and a second slot 30b′ starting from the side S2 of the fastening flange 2″ opposite to the front side VS, which end in a respectively associated first broadening region 31a′ and second broadening region 31b′.

The rectilinear region 25 of the cutting guide attachment 200′ has a first and second locking bolt 210a, 210b, which can be introduced through the associated slot 30a′, 30b′ into the first and second broadening regions 31a′, 31b′ in order to lock the ring profile.

Additional guide ridges 33a, 33b on the side S2 make it easier to fit the cutting guide attachment 200′ on. Locking may be achieved by using the material properties of the plastic.

In other regards, the sixth embodiment is formed in entirely the same way as the first embodiment.

FIG. 10 shows a partial perspective exploded view of a resection template according to a seventh embodiment of the present invention.

The representation of FIG. 10 is similar to the representation of FIG. 2, and differs from FIG. 2 in respect of the configuration of the cutting guide attachment 200″ and of the fastening flange 2″″ as well as the contour of the base part 1″.

In particular, the fastening flange 2″″ is formed as a spiked flange having a spike. The cutting guide attachment 200″ is formed as a ring profile, which has a spike reception region A′ and a saw abutment 200a′.

The spike and the spike reception region have an asymmetrical round profile, which prevents fitting on incorrectly.

In other regards, the seventh embodiment is formed in entirely the same way as the first embodiment.

FIG. 11 shows a partial perspective representation of a resection template according to an eighth embodiment of the present invention.

The representation of FIG. 11 is similar to the representation of FIG. 5b), and differs from FIG. 5b) in respect of the configuration of the cutting guide attachment 200′″ and of the fastening flanges 2″″a, 2″″b as well as the contour of the base part 1′″.

In particular, the fastening flanges 2″″a, 2″″b are formed as spiked flanges having a respective spike. The cutting guide attachment 200′″ is formed as a ring profile, which has a respective spike reception region A″, A′″ and a saw slot 201. The saw slot 201 can be introduced along a respective slot SLa, SLb of the base part 1′″.

The spikes and the associated spike reception regions have a respective asymmetrical round profile, which prevents fitting on incorrectly.

In other regards, the eighth embodiment is formed in entirely the same way as the second embodiment.

FIG. 12 shows a partial perspective representation of a resection template according to a ninth embodiment of the present invention.

In the ninth embodiment, the fastening flange 2′a does not have broadening regions and end abutment regions. The front side of the base part 1″, which has the abutment block 22c that is arranged in such a way that it forms a respective lateral abutment for the first and second end collar regions 25a′, 25b′ of the cutting guide attachment 200″, is used as an end abutment.

A groove 39 is provided on the side S2 of the fastening flange 2′a opposite to the front side VS, the cutting guide attachment 200″″ having a connecting region V, which can be inserted into the groove 39, starting from the rectilinear region 25, and a grip region GB being provided on the region of the connecting region V opposite to the rectilinear region 25.

In other regards, the ninth embodiment is formed in entirely the same way as the fourth embodiment.

FIG. 13a), b) show perspective representations of a resection template according to a tenth embodiment of the present invention, specifically with FIG. 13a) showing a partial perspective representation with a fitted saw tool in a first position of the saw tool and FIG. 13b) showing a partial perspective representation with a fitted saw tool in a second position of the saw tool.

The tenth embodiment differs from the first embodiment in the configuration of the cutting guide attachment 20a. In particular, in the above-described embodiments of the cutting guide attachment, the upper side and the lower side of the cutting guide attachment are arranged plane-parallel. This has the effect that the cutting depth of the saw tool fitted on is constant over the entire lateral extent of the cutting guide attachment.

In the tenth embodiment, however, the upper side of the cutting guide attachment 20a has a curved contour KON, the effect of which is that the cutting depth of the saw tool fitted on varies over the lateral extent of the cutting guide attachment 20a.

In the tenth embodiment, the contour KON of the upper side of the cutting guide attachment 20a is curved in such a way that it replicates or at least locally approximates a contour KON′ of the underlying bone KN to be resected. In this way, tissue structures which surround the bone KN to be resected may be protected since they cannot be reached by the saw tool S.

In other regards, the tenth embodiment is formed in entirely the same way as the first embodiment.

Of course, such a configuration of the cutting guide attachment with a curved contour of the upper side may also be produced in all other embodiments described above.

Although the present invention has been explained with the aid of preferred embodiments, it is not restricted thereto but may be modified in a variety of ways, particularly in respect of the geometry and materials of its constituents.

1. A resection template for the resection of a bone, having:
a plate-shaped base part having a front side and a rear side;
wherein the rear side has a bearing face for bearing on the bone;
wherein the front side has a fastening flange which extends away from the front side and is arranged next to an end edge of the base part or a slot of the base part;
a cutting guide attachment which can be fitted onto the fastening flange from a side of the fastening flange opposite to the front side;
wherein the cutting guide attachment has a saw slot or a saw abutment for guiding a sawing tool, which slot or abutment is arranged along the end edge of the base part or along the slot of the base part when the cutting guide attachment is fitted on.

2. The resection template according to claim 1, wherein the fastening flange is formed as a plate flange.

3. The resection template according to claim 2, wherein the cutting guide attachment is formed as a ring profile, which has a rectilinear region in which the saw slot is arranged or on which the saw abutment is arranged, wherein there is a first end collar region at the first end of the rectilinear region and a second end collar region at the second end of the rectilinear region, wherein the first and second end collar regions together with the rectilinear region form a reception region for the plate flange, and wherein the first and second end collar regions are formed in such a way that they enclose the plate flange on its opposite edge regions.

4. The resection template according to claim 3, wherein there is an open region of the ring profile between the first and second end collar regions.

5. The resection template according to claim 4, wherein the plate flange has a first abutment ridge and a second abutment ridge, which are arranged in such a way that they form a respective lateral abutment for the first and second end collar regions.

6. The resection template according to claim 4, wherein the plate flange has an abutment block, which is arranged in such a way that it forms a respective lateral abutment for the first and second end collar regions.

7. The resection template according to claim 2, wherein the plate flange has on its opposite edges a respective first and second broadening region, which widen in the direction from the side of the plate flange opposite to the front side in the direction of the front side, so that an increasing clamp effect can be exerted on the ring profile when fitting on.

8. The resection template according to claim 2, wherein the plate flange has on its opposite edges a respective first and second end abutment region, which limit the fitting depth of the ring profile onto the plate flange.

9. The resection template according to claim 3, wherein the first and second end collar regions have a respective gripping rib region.

10. The resection template according to claim 3, wherein the plate flange has a first slot and a second slot starting from the side of the plate flange opposite to the front side, which end in a respectively associated first broadening region and second broadening region, and wherein the first and second end collar regions have a first and second locking bolt, which can be introduced through the associated slot into the first and second broadening regions in order to lock the ring profile.

11. The resection template according to claim 3, wherein the plate flange has a first slot and a second slot starting from the side of the plate flange opposite to the front side, which end in a respectively associated first broadening region and second broadening region, and wherein the rectilinear region has a first and second locking bolt, which can be introduced through the associated slot into the first and second broadening regions in order to lock the ring profile.

12. The resection template according to claim 1, wherein the fastening flange is formed as a spiked flange having one or more spikes.

13. The resection template according to claim 12, wherein the cutting guide attachment is formed as a ring profile which has one or more spike reception regions, according to the number of spikes of the spiked flange.

14. The resection template according to claim 3, wherein the plate flange has a groove on the side of the plate flange opposite to the front side, wherein the cutting guide attachment has a connecting region, which can be inserted into the groove, starting from the rectilinear region, and wherein a grip region is provided on the region of the connecting region opposite to the rectilinear region.

15. The resection template according to claim 1 one of the preceding claims, wherein the plate-shaped base part has a multiplicity of through-holes, which are used to receive fastening means, in particular screws or nails, for fastening on the bone.

16. The resection template according to claim 1 one of the preceding claims, wherein the plate-shaped base part with the fastening flange is formed from a plastic material and the cutting guide attachment is formed from a metallic material.

17. The resection template according to claim 1, wherein the cutting guide attachment is formed in such a way that the upper side and the lower side of the cutting guide attachment are arranged plane-parallel, so that the cutting depth of a saw tool fitted on is constant over the entire lateral extent of the cutting guide attachment.

18. The resection template according to claim 1, wherein the cutting guide attachment is formed in such a way that the upper side of the cutting guide attachment has a curved contour, the effect of which is that the cutting depth of a saw tool fitted on varies over the lateral extent of the cutting guide attachment.

19. The resection template according to claim 18, wherein the curved contour of the upper side of the cutting guide attachment is curved in such a way that it replicates or at least locally approximates a contour of the underlying bone to be resected.
